(19) United States
(12) Patent Application Publication
(10) Pub. No.: US 2006/0247717 A1
(43) Pub. Date: Nov. 2, 2006

(54) Electrical stimulation of the gastrointestinal tract to regulate motility

(75) Inventor: Warren L. Starkebaum, Plymouth, MN (US)

Correspondence Address: SHUMAKER & SIEFFERT, P. A., 8425 SEASONS PARKWAY, SUITE 105, ST. PAUL, MN, 55125, US

(73) Assignee: Medtronic, Inc., Minneapolis, MN (US)

(21) Appl. No.: 11/116,951

(22) Filed: Apr. 28, 2005

(51) Int. Cl. A61N 1/18

(52) U.S. Cl. 607/40

(57) ABSTRACT

The disclosure describes a therapeutic gastric stimulation system that regulates gastric motility. The system delivers electrical stimulation pulses in the range of 30 to 120 pulses per minute to substitute for inadequate gastric spike action potentials. The delivery of stimulation pulses to mimic gastric spike activity may enable increased motility as therapy for gastroparesis. Pulses may be delivered continuously without feedback, in bursts without feedback, or in bursts synchronized to the patient's intrinsic gastric slow wave.

### TECHNICAL FIELD

The invention relates to implantable medical devices and, more particularly, implantable gastric stimulators.

### BACKGROUND

Gastroparesis is an adverse medical condition in which normal gastric motor function is impaired, undermining proper gastric motility. Gastroparesis results in delayed gastric emptying as the stomach cannot move its contents at a normal rate. Typically, gastroparesis results when muscles within the stomach or intestines are not working normally, resulting in a stoppage or slowdown in movement of food through the stomach. Patients with gastroparesis typically exhibit symptoms of nausea and vomiting, as well as gastric discomfort such as bloating or a premature or extended sensation of fullness, i.e., satiety. Gastroparesis generally causes reduced food intake and subsequent weight loss, and can adversely affect patient health.

The causes of decreased gastric motility may be varied. In some cases, disease may disrupt the ability of nerves to communicate stimulation information to the smooth muscle in the stomach wall. Some patients may develop decreased motility after undergoing a surgical procedure. In other cases, major trauma to the nervous system or digestive system may impair motility. In all cases, gastroparesis is a serious disorder that can adversely affect the health and quality of life of a patient.

Electrical stimulation of the gastrointestinal tract has been used to treat symptoms of gastroparesis. For example, electrical stimulation of the gastrointestinal tract, and especially the stomach, is effective in suppressing symptoms of nausea and vomiting secondary to diabetic or idiopathic gastroparesis. Typically, electrical stimulation involves the use of electrodes implanted in the wall of a target organ. The electrodes are electrically coupled to an implanted or external pulse generator via implanted or percutaneous leads. The pulse generator delivers a stimulation waveform via the leads and electrodes. Depending on the condition of the patient, this therapy also may be successful in increasing gastric motility.

### SUMMARY

The invention is directed to techniques for regulating gastrointestinal motility by electrical stimulation of the gastrointestinal tract. The electrical stimulation is delivered as a set of stimulation pulses at a rate of approximately 30 to 120 pulses per minute to mimic the frequency of gastric “spike” activity that ordinarily accompanies a normal gastric slow wave in a healthy patient. By targeting the spike activity linked to peristaltic contraction, this “spike stimulation” can enhance gastric motility, reduce symptoms of nausea, and reduce premature or extended satiety for patients suffering from gastroparesis or other gastrointestinal motility disorders.

The stimulation pulses may be delivered in a variety of different modes, such as a continuous mode, an asynchronous burst mode, or a synchronous burst mode. In a continuous mode, the pulse train is delivered relatively continuously. In the asynchronous burst mode, the pulse train is delivered in periodic bursts. In the synchronous burst mode, the pulse train is delivered in bursts that are synchronized with a sensed event, such as a sensed gastric slow wave. Each mode may be activated on a full-time basis, or for selected parts of a day, such as periods of time coinciding with meals.

In one embodiment, the invention provides a method for electrical stimulation of a gastrointestinal tract of a patient, the method comprising generating electrical stimulation pulses at a rate of approximately 30 to 120 pulses per minute, and delivering the stimulation pulses to the gastrointestinal tract to regulate gastric motility.

In another embodiment, the invention provides a system for electrical stimulation of a gastrointestinal tract of a patient, the system comprising a pulse generator that generates electrical stimulation pulses at a rate of 30 to 120 pulses per minute, and one or more leads that apply the pulses to the gastrointestinal tract to regulate gastric motility.

In an additional embodiment, the invention provides a system for electrical stimulation of a gastrointestinal tract of a patient, the system comprising means for generating electrical stimulation pulses at a rate of approximately 30 to 120 pulses per minute, and means for delivering the stimulation pulses to the gastrointestinal tract to regulate gastric motility.

In a further embodiment, the invention provides a method for electrical stimulation of a gastrointestinal tract of a patient, the method comprising, generating electrical stimulation pulses at a rate of approximately 30 to 120 pulses per minute, delivering the pulses to the gastrointestinal tract in bursts at a rate of approximately 2 to 20 bursts per minute to regulate gastric motility.

In various embodiments, the invention may provide one or more advantages. For example, the delivery of electrical stimulation to mimic gastric spike activity may more effectively promote gastric motility. By targeting the spike activity ordinarily associated with peristaltic movement, the spike stimulation frequency range may provide for faster movement of food through the gastrointestinal tract. In this manner, the patient does not need to rely only on gastric slow wave stimulation to induce contractions of smooth muscle, e.g., within the stomach wall. Rather, the spike stimulation more aggressively targets gastric motility. The invention may provide a straightforward and energy efficient approach to stimulating the gastrointestinal tract to improve motility for dysmotility conditions such as gastroparesis or post operative ileus. In addition, the invention may be applicable to treatment of non-dysmotility conditions such as obesity. In particular, the spike stimulation may be applied to increase motility to reduce caloric absorption.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic diagram illustrating an implantable stimulation system for regulating gastric motility by delivering stimulation that mimics gastric spike activity.

FIG. 2 is a functional block diagram illustrating various components of an exemplary implantable stimulator.

FIG. 3 is a chart illustrating a normal gastric slow wave and pulses delivered continuously during asynchronous stimulation therapy to mimic gastric spike activity.

FIG. 4 is a chart illustrating a dysfunctional gastric slow wave and bursts of pulses delivered during asynchronous burst stimulation therapy to mimic gastric spike activity.

FIG. 5A is a timing diagram illustrating the programming of a pulse generator to deliver a series of stimulation pulses at a rate selected to mimic gastric spike activity.

FIG. 5B is a timing diagram illustrating the programming of a pulse generator to deliver bursts of stimulation pulses.

FIG. 6 is a functional block diagram illustrating various components of an exemplary implantable stimulator with sensing capabilities.

FIG. 7 is a chart of a normal gastric slow wave and synchronized busts of pulses delivered during synchronous burst stimulation therapy.

FIG. 8 is a flow chart illustrating a technique for delivery of synchronous burst stimulation therapy on a closed loop basis in response to the sensing of an intrinsic gastric slow wave.

### DETAILED DESCRIPTION

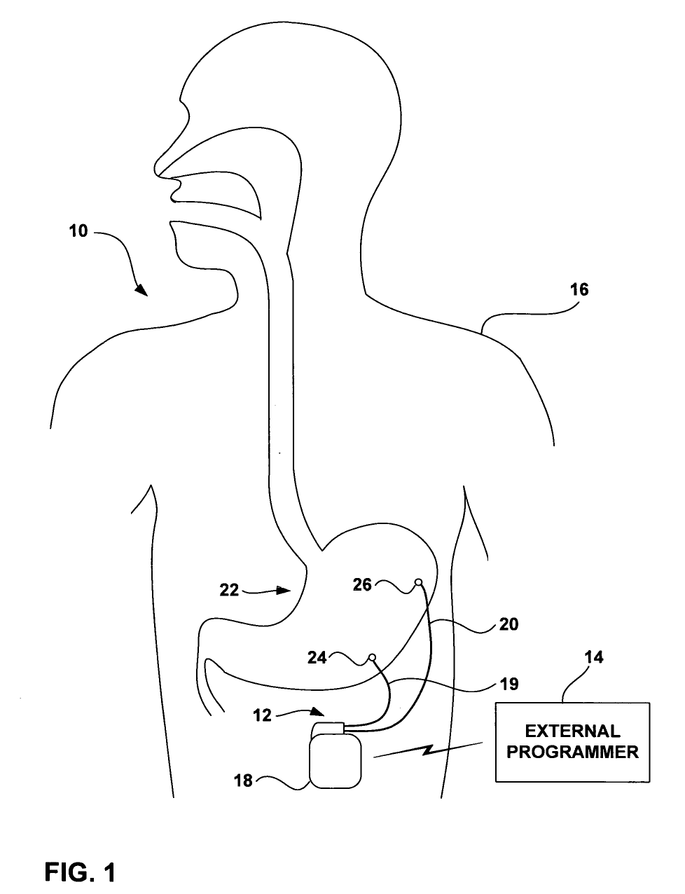

FIG. 1 is a schematic diagram illustrating an implantable stimulation system 10. System 10 may be configured to deliver therapy for alleviation of gastroparesis. As shown in FIG. 1, system 10 may include an implantable stimulator 12 and external programmer 14 shown in conjunction with a patient 16. Stimulator 12 includes a pulse generator 18 that generates electrical stimulation pulses. One or more leads 19, 20 carry the electrical stimulation pulses to stomach 22. Although the electrical stimulation pulses may be delivered to other areas within the gastrointestinal tract, such as the esophagus, duodenum, small intestine, or large intestine, delivery of stimulation pulses to stomach 22 will generally be described in this disclosure for purposes of illustration.

At the surface lining of stomach 22, leads 19, 20 terminate into tissue at electrodes 24 and 26, respectively. The stimulation pulses generated by stimulator 12 cause the smooth muscle of stomach 22 to contract and slowly move contents from the entrance toward the exit of the stomach. Alternatively, or additionally, the electrical stimulation pulses may stimulate nerves within stomach 22 to cause muscle contraction and thereby restore or enhance gastrointestinal motility. Again, the stimulation pulses may be delivered elsewhere within the gastrointestinal tract, either as an alternative to stimulation of stomach 22 or in conjunction with stimulation of the stomach. In addition, system 10 may be applicable to treatment of non-dysmotility conditions such as obesity. In particular, the spike stimulation may be applied to increase motility to reduce caloric absorption.

Implantable stimulator 12 may be constructed with a biocompatible housing, such as titanium, stainless steel, or a polymeric material, and is surgically implanted within patient 16. The implantation site may be a subcutaneous location in the side of the lower abdomen or the side of the lower back. Pulse generator 18 is housed within the biocompatible housing, and includes components suitable for generation of electrical stimulation pulses. Electrical leads 19 and 20 are flexible, electrically insulated from body tissues, and terminated with electrodes 24 and 26 at the distal ends of the respective leads. The leads may be surgically or percutaneously tunneled to stimulation sites on stomach 22. The proximal ends of leads 19 and 20 are electrically coupled to pulse generator 18 to conduct the stimulation pulses to stomach 22.

Leads 19, 20 may be placed into the muscle layer or layers of stomach 22 via an open surgical procedure, or by laparoscopic surgery. Leads also may be placed in the mucosa or submucosa by endoscopic techniques, or by an open surgical procedure or laparoscopic surgery. Electrodes 24, 26 may form a bipolar pair of electrodes. Alternatively, pulse generator 18 may carry a reference electrode to form an “active can” arrangement, in which electrodes 24, 26 are unipolar electrodes referenced to the electrode on the pulse generator. A variety of polarities and electrode arrangements may be used.

In accordance with the invention, the electrical stimulation pulses are delivered at a rate of approximately 30 to 120 pulses per minute to mimic the spike activity that ordinarily accompanies a normal gastric slow wave in a healthy patient. By targeting the spike activity linked to peristaltic contraction, this “spike stimulation” can enhance gastric motility, and reduce symptoms of nausea, vomiting, early satiety and bloating associated with gastroparesis. Optionally, the spike stimulation may be delivered as bursts of pulses with a burst rate of approximately 2 to 20 pulses per minute. The pulses in each burst are delivered at a frequency selected to mimic spike activity, while each burst is delivered at a frequency selected to mimic slow wave activity.

The pulse train may be delivered in a variety of different modes, such as a continuous mode, an asynchronous burst mode, or a synchronous burst mode. In a continuous mode, the pulse train is delivered relatively continuously over an active period in which stimulation is “ON.” In an asynchronous burst mode, the pulse train is delivered in periodic bursts during the active period. The continuous mode and asynchronous burst mode may be considered open loop in the sense that they do not rely on synchronization with sensed events, such as the intrinsic gastric slow wave.

In the synchronous burst mode, the pulse train is delivered in bursts that are synchronized with a sensed event, such as a sensed gastric slow wave. In this sense, the synchronous burst mode may be viewed as a closed loop approach. The active period for each mode may be full-time, part-time, or subject to patient control. For part-time activation, the stimulation may be activated for selected parts of the day. The selected parts of the day may coincide with meal times, physical activity times, sleep times, or other selected times, and be controlled using a clock within pulse generator 18 or programmer 14.

In addition to pulse rate, the stimulation pulses delivered by stimulator 12 are characterized by other stimulation parameters such as a voltage or current amplitude and pulse width. The stimulation parameters may be fixed, adjusted in response to sensed physiological conditions within or near stomach 22, or adjusted in response to patient input entered via external programmer 14. For example, in some embodiments, patient 16 may be permitted to adjust stimulation amplitude and turn stimulation on and off.

In addition, as mentioned above, the timing of stimulation may be controlled in a synchronous mode in response to sensed intrinsic slow wave activity. To sense intrinsic slow wave activity, implantable stimulator 12 may be equipped to sense the intrinsic gastric slow wave, if present, and control the delivery of stimulation pulses in response to the gastric slow wave.

One or both of leads 19, 20 may carry a sense electrode, in addition to stimulation electrodes, to sense the intrinsic gastric slow wave. Alternatively, an additional lead or device may be provided to sense the intrinsic gastric slow wave. Sensing may occur continuously, periodically, or intermittently, as therapy dictates. Information relating to the sensed intrinsic gastric slow wave signals may be stored in memory within pulse generator 18 for retrieval and analysis at a later time.

Pulse generator 18 also may include telemetry electronics to communicate with external programmer 14. External programmer 14 may be a small, battery-powered, portable device that accompanies patient 16 throughout a daily routine. Programmer 14 may have a simple user interface, such as a button or keypad, and a display or lights. External programmer 14 may be a hand-held device configured to permit activation of stimulation and adjustment of stimulation parameters. Alternatively, programmer 14 may form part of a larger device including a more complete set of programming features including complete parameter modifications, firmware upgrades, data recovery, or battery recharging in the event stimulator 12 includes a rechargeable battery.

In some embodiments, system 10 may include multiple implantable stimulators 12 to stimulate a variety of regions of stomach 22. Stimulation delivered by the multiple stimulators may be coordinated in a synchronized manner, or performed without communication between stimulators. Also, the electrodes may be located in a variety of sites on the stomach dependent on the particular therapy or the condition of patient 12.

The electrodes carried at the distal end of each lead 19, 20 may be attached to the wall of stomach 22 in a variety of ways. For example, the electrode may be surgically sutured onto the outer wall of stomach 22 or fixed by penetration of anchoring devices, such as hooks, barbs or helical structures, within the tissue of stomach 22. Also, surgical adhesives may be used to attach the electrodes. In any event, each electrode is implanted in acceptable electrical contact with the smooth muscle cells within the wall of stomach 22. In some cases, the electrodes may be placed on the serosal surface of stomach 22, within the muscle wall of the stomach, or within the mucosal or submucosal region of the stomach.

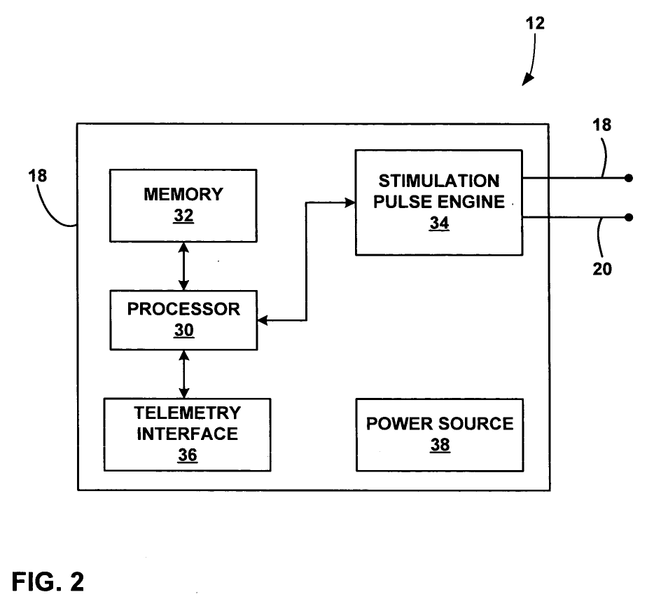

FIG. 2 is a functional block diagram illustrating various components of an exemplary implantable stimulator 12. Stimulator 12 includes a pulse generator 18 including a processor 30, memory 32, stimulation pulse engine 34, telemetry interface 36, and power source 38. Electrical leads 19 and 20 extend from the housing and terminate at stomach 22. Memory 32 stores instructions for execution by processor 30, stimulation parameters and, optionally, sense information relating to sensed physiological conditions. Memory 32 may include separate memories for storing instructions, stimulation parameter sets, and stimulation information, or a common memory.

Pulse generator 18 may generally conform to the pulse generator provided in the Enterra Therapy™ Gastric Electrical Stimulation (GES) System, manufactured by Medtronic, Inc. of Minneapolis, Minn. For operation, pulse generator 18 is programmed with stimulation pulse parameters appropriate for delivery of spike stimulation in the form of stimulation pulses delivered continuously at a rate of approximately 30 to 120 pulses per minute, or delivered as bursts of stimulation pulses at a rate of 2 to 20 bursts per minute to mimic slow wave activity. Within each burst, the pulses may be delivered at a rate of approximately 30 to 120 pulses per minute.

Processor 30 controls stimulation pulse engine 34 to deliver electrical stimulation therapy. Based on stimulation parameters programmed by external programmer 14, processor 30 instructs appropriate stimulation by stimulation pulse engine 34. Information may be received from external programmer 14 at any time during operation, in which case a change in stimulation parameters may immediately occur. Processor 30 determines any pulse parameter adjustments based on the received information, and loads the adjustments into memory 32 for use during delivery of stimulation.

Wireless telemetry in stimulator 12 may be accomplished by radio frequency (RF) communication or proximal inductive interaction of implantable stimulator 12 with external programmer 26 via telemetry interface 36. Processor 30 controls telemetry interface 36 to exchange information with external programmer 14. Processor 30 may transmit operational information and sensed information to programmer 14 via telemetry interface 36. Also, in some embodiments, pulse generator 18 may communicate with other implanted devices, such as stimulators or sensors, via telemetry interface 26.

Power source 38 delivers operating power to the components of implantable stimulator 12. Power source 38 may include a battery and a power generation circuit to produce the operating power. In some embodiments, the battery may be rechargeable to allow extended operation Recharging may be accomplished through proximal inductive interaction between an external charger and an inductive charging coil within stimulator 12. In other embodiments, an external inductive power supply may transcutaneously power stimulator 12 whenever stimulation therapy is to occur.

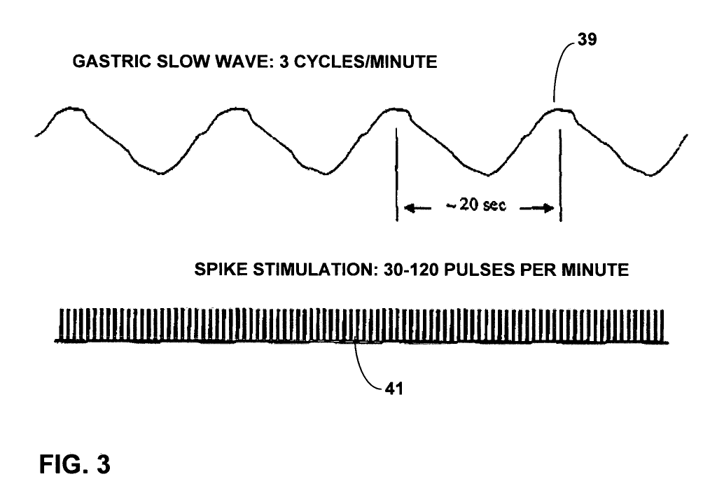

FIG. 3 is a chart showing an exemplary intrinsic gastric slow wave 39 and a series of stimulation pulses 41 delivered in a continuous, asynchronous mode to mimic gastric spike activity. The gastric slow wave 39 shown is a normal gastric slow wave signal in a healthy patient. This slow wave 39 is a steady electrical rhythm that occurs at approximately three cycles per minute, or one cycle approximately every 20 seconds. In a healthy patient, this gastric slow wave 41 is always present, even without food present in stomach 22 and in the absence of peristalsis. Most patients retain a healthy slow wave, but may suffer from the inability to promote normal motility due to absent or abnormal gastric spike action potentials.

When peristalsis occurs in stomach 22 after food is ingested, for example, spike action potentials can be observed in healthy patients superimposed on the slow wave. The spike action potentials are indicative of the smooth muscle contraction of peristalsis. In effect, the slow wave does not cause smooth muscle contractions to occur, but instead regulates the rate at which bursts of spike action potentials occur in stomach 22. Gastric spike action potentials are indicative of depolarization of smooth muscle and contractions that result in peristalsis. In some patients, poor gastric motility may result from slow, fast, or irregular slow waves combined with inadequate spike potentials.

In the mode of therapy described in FIG. 3, stimulation pulses are delivered in an asynchronous, continuous mode to mimic gastric spike activity. The stimulation pulses may be delivered by an implanted stimulator 12 similar to that illustrated in FIGS. 1 and 2. The spike stimulation includes electrical pulses delivered to smooth muscle continuously at a frequency between 30 and 120 pulses per minute, which corresponds to an inter-pulse interval of approximately 0.5 to 2.0 seconds. The terms “rate” and “frequency” are used interchangeably in this disclosure.

In the example of FIG. 3, stimulator 12 delivers the stimulation pulses 41 continuously on an open loop basis, without any feedback of sensed events or conditions. In addition, the pulses 41 are not synchronized to the frequency of the gastric slow wave 39, or synchronized to the phase of the slow wave at a certain time. The superposition of the stimulation pulses 41 over the slow wave causes the membrane potential of the smooth muscle to reach threshold and depolarize, thus inducing smooth muscle contractions and motility.

The stimulation pulses are delivered at a rate of approximately 30 to 120 pulses per minute, and more preferably 60 to 90 pulses per minute. The 30 to 120 pulse per minute range corresponds to an inter-pulse interval of approximately 0.5 to 2.0 seconds, while the 60 to 90 pulse per minute range corresponds to an inter-pulse interval of approximately 1.0 to 0.67 seconds. The stimulation pulses 41 may be delivered with a voltage amplitude in a range of approximately 1 to 15 volts, and more preferably 2 to 7.5 volts. Each stimulation pulse may have a pulse width of approximately 0.05 to 10 milliseconds, and more preferably 0.1 to 0.5 milliseconds.

As mentioned previously, stimulation parameters may be modified before, after, or during stimulation therapy. In the case of parameter modifications during therapy, the stimulation may be changed immediately for the next pulse or the stimulation may gradually change to meet the new stimulation levels. A gradual change may be used to guard against abrupt tissue stimulation increases or decreases that may negatively affect the motility of stomach 22.

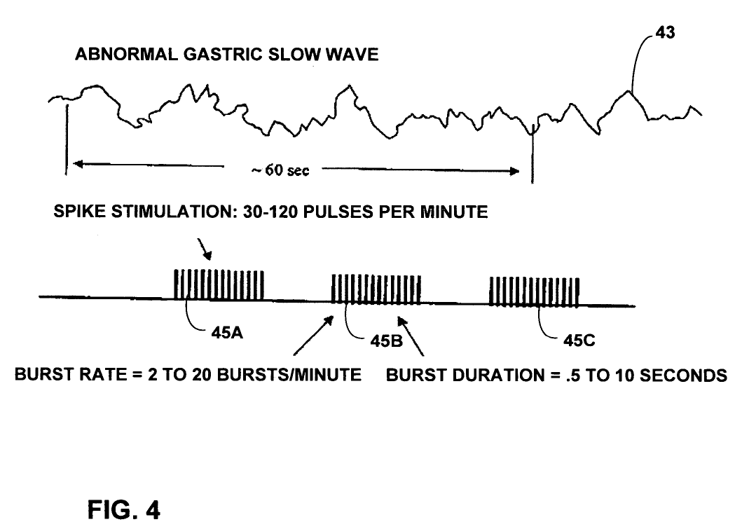

FIG. 4 is a chart showing a dysfunctional gastric slow wave 43 and bursts 45A, 45B, 45C (collectively 45) of stimulation pulses delivered in an asynchronous burst mode to mimic gastric spike activity. In some cases, the patient may have a dysfunctional or nonexistent gastric slow wave. The gastric slow wave 43 shown in FIG. 4 is an exemplary dysfunctional gastric signal that may have no discernable frequency or amplitude. In this case, patient 16 may have limited gastric motility due to the abnormal and erratic electrical activity.

In accordance with some embodiments, stimulation pulses are delivered in bursts 45, as shown in FIG. 4, to mimic spike activity that is normally observed superimposed on the gastric slow wave. Stimulation bursts 45 are delivered at rates selected to mimic the frequency of a normal slow wave, particularly for patients suffering from a dysfunctional or nonexistent slow wave. However, the stimulation pulses within each burst 45 are delivered at rates selected to mimic spike activity.

Similar to the asynchronous, continuous stimulation described with respect to FIG. 3, for example, the electrical stimulation pulses within each burst 45 are delivered at a rate of approximately 30 to 120 pulses per minute, and more preferably approximately 60 to 90 pulses per minute. Again, these rates correspond to an inter-pulse interval of approximately 0.5 to 2.0 seconds, and approximately 1.0 to 0.67 seconds, respectively. The amplitudes and pulse widths of the pulses in bursts 45 may be similar to those described above with respect to the continuous mode stimulation pulses 41 of FIG. 3. However, the pulses are not delivered continuously. Instead, the stimulation pulses are delivered in bursts 45, as shown in FIG. 4.

Each burst 45 of stimulation pulses may occurs at a rate of approximately 2 to 20 bursts per minute, and more preferably approximately 2.7 to 3.5 bursts per minute, with the duration of each burst being in a range of approximately 0.5 to 10 seconds, and more preferably approximately 2 to 5 seconds. Successive bursts 45 may be separated by a fixed time interval, which may be programmed into stimulator 12. Stimulator 12 delivers the bursts 45 of stimulation pulses on an open loop basis, i.e., without feedback from a sensor. The pulses are not synchronized to the specific frequency of the gastric slow wave, if one would be detectable. The superposition of the stimulation pulses over background neuronal activity may cause the membrane potential of the smooth muscle to reach threshold and depolarize, thus inducing smooth muscle contractions and enhanced motility.

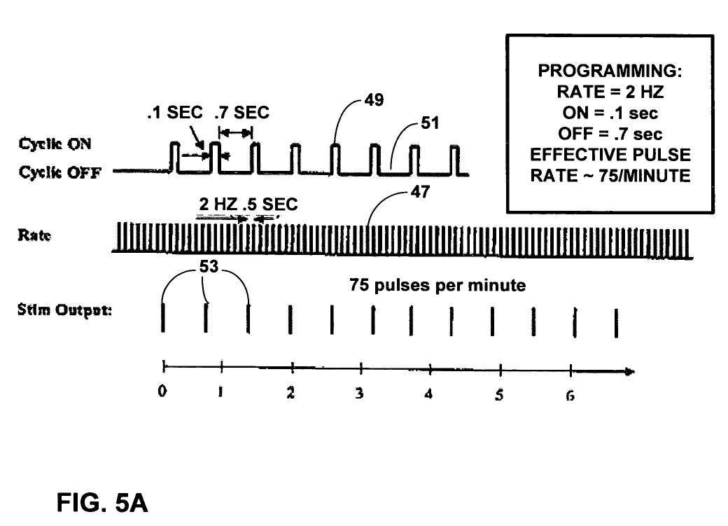

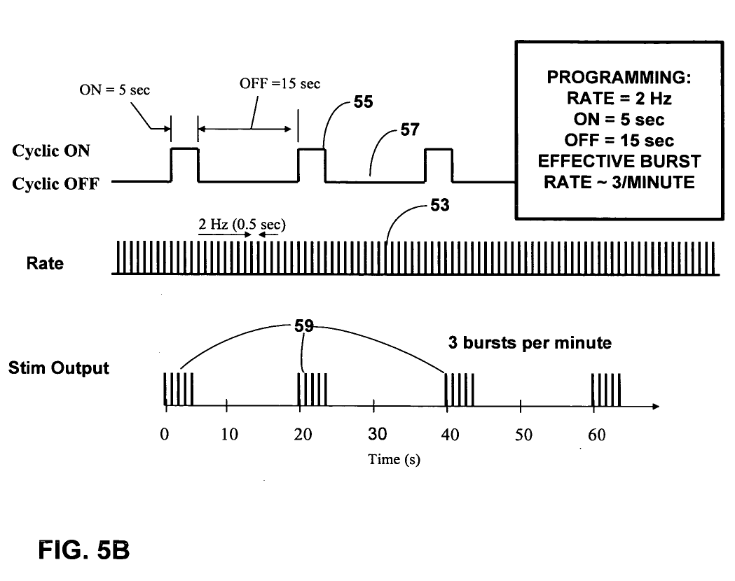

FIG. 5A is a timing diagram illustrating programming of pulse generator 18 to deliver a continuous train of stimulation pulses to mimic spike activity. For example, the diagram of FIG. 5 illustrates how a pulse generator provided in the Enterra Therapy™ Gastric Electrical Stimulation (GES) System, manufactured by Medtronic, Inc. of Minneapolis, Minn. In the example of FIG. 5A, pulse generator 18 is programmed to output a continuous train of stimulation pulses 53 at a rate selected to mimic gastric spike activity. The pulse generator 18 may be programmed to produce a pulse train 47 at a frequency (F) of 2 Hz. Pulse train 47 is then cycled by specifying “ON” and “OFF” cycles 49, 51, respectively. In the example of FIG. 5, pulse train 47 is cycled ON for 0.1 seconds and cycled OFF for 0.7 seconds. The result is the delivery of a series of stimulation pulses 53 at a rate of approximately 75 per minute.

FIG. 5B is a timing diagram illustrating programming of pulse generator 18 to deliver asynchronous bursts of stimulation pulses. Like FIG. 5A, FIG. 5B illustrates how a pulse generator may be programmed. However, FIG. 5B illustrates programming of pulse generator 18 to deliver asynchronous bursts of pulses where each burst consists of a series of pulses. In the example of FIG. 5B, pulse generator 18 is programmed to produce a train of stimulation pulses 53, as in FIG. 5A. In FIG. 5B, a train of stimulation pulses 53 is delivered at a frequency of approximately 2 Hz, i.e., 120 pulses per minute. In FIG. 5B, however, pulses 53 are not delivered continuously as in the example of FIG. 5A. Instead, pulses 53 are gated by an additional ON cycle 55 and OFF cycle 57 to produce bursts 59 of pulses 53.

In particular, the train of pulses 53 is cycled ON for 5 seconds and cycled OFF for 15 seconds. The result is the delivery of a series of bursts 59 of stimulation pulses 53 at a rate of approximately 3 bursts per minute. Each burst 61 has a burst length of approximately 5 seconds and contains several individual stimulation pulses 53. Thus, adjusting pulse frequency, cycle ON and Cycle OFF times can be used to produce various pulse and burst frequencies, and burst durations. Each burst 61 contains pulses 53 delivered at a rate selected to mimic the spike activity, while the bursts are delivered at a rate selected to mimic gastric slow wave activity. In the example of FIG. 5B, each burst 61 contains pulses 53 delivered at 120 pulses per minute, and each burst 61 is delivered at 3 bursts per minute. Other values and rates within the ranges described herein may be used.

In other embodiments, different pulse generators may be used to create the same effective frequency of stimulation pulses to mimic spike activity. In addition, programming may involve setting of other parameters to configure the pulse generator 18 for proper therapy. For example, programming pulse generator 12 may involve selecting interval times between bursts, burst durations, pulse amplitude, pulse width, therapy duration, and active periods during which the pulse generator operates. The therapy duration may span several minutes, hours, days, weeks or years, while active periods may specify particular periods of time, over the course of the therapy duration, in which stimulator 12 is active. The active periods may be time to coincide with meals, or be indicated by patient 16 when necessary after ingesting food.

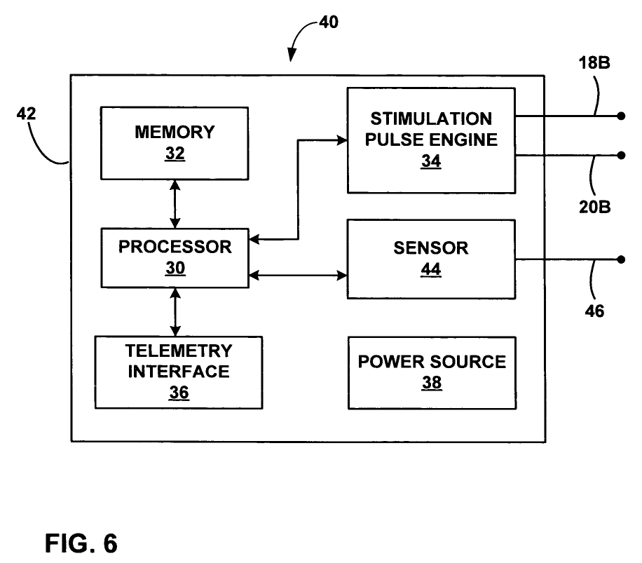

FIG. 6 is a functional block diagram illustrating various components of an implantable stimulator 40 that may be used to provide synchronous burst stimulation. Stimulator 40 may generally conform to stimulator 12 of FIG. 2. For example, stimulator 40 of FIG. 6 includes a pulse generator 42, which incorporates a processor 30, memory 32, stimulation pulse engine 34, telemetry interface 36, and power source 38. In addition, stimulator 40 includes electrical leads 19 and 20, which extend from the housing and terminate at or near stimulation sites within stomach 22 or other areas within the gastrointestinal tract.

Pulse generator 42 further includes, however, a sensor 44 coupled to a sensor lead 46. Sensor lead 46 may carry an electrode to sense electrical potentials within the gastrointestinal tract. In particular, sensor 44 and sensor lead 46 may be positioned to sense the intrinsic gastric slow wave within the gastrointestinal tract of patient 16. In the example of FIG. 6, the sensed signal is processed by processor 30 in order to synchronize the generation of bursts of stimulation pulses to be delivered to patient 16.

Synchronization may be in the form of bursts of stimulation pulses gated according to a particular threshold amplitude of the gastric slow wave. In some embodiments, a burst of spike stimulation pulses may be delivered in substantial synchronization with a peak amplitude of the gastric slow wave. Upon detection of the peak via sensor 44 and lead 46, processor 30 enables stimulation pulse engine 34 to deliver a burst of stimulation pulses to mimic spike activity. In this manner, stimulator 40 supports delivery of spike stimulation pulses in a synchronous mode that is responsive to a sensed gastric slow wave.

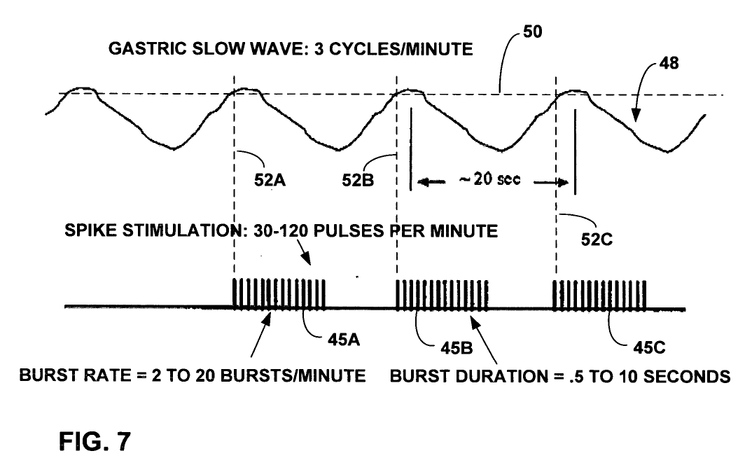

FIG. 7 is a chart showing an intrinsic gastric slow wave in conjunction with delivery of stimulation pulses in a synchronous burst mode to mimic spike activity. The chart of FIG. 7 provides an example of the operation of stimulator 40 of FIG. 6. The gastric slow wave 48 shown in FIG. 7 is a normal gastric slow wave at a steady electrical rhythm, occurring at approximately three cycles per minute, or one cycle every 20 seconds. In the synchronous burst mode of therapy illustrated in FIG. 7, synchronous bursts 45A, 45B, 45C are delivered in synchronization with an aspect of the gastric slow wave, such as the crossing of a threshold 50. When the gastric slow wave 48 crosses threshold 50, for example, stimulator 40 triggers delivery of spike stimulation in the form of bursts 45 at respective times indicated by reference numerals 52A, 52B, 52C. The stimulation pulses in each burst 45 are delivered at a rate in a range of approximately 30 and 120 pulses per minute, i.e., at an inter-pulse interval of approximately 0.5 to 2.0 seconds, to mimic spike activity. Each burst 45 may have a duration in a range of approximately 0.5 to 10 seconds.

As shown in FIG. 7, stimulator 40 delivers bursts of pulses on a closed loop basis, in synchronization with slow wave 48, e.g., as determined by feedback from sensor 44 (FIG. 6). The sensor 44 may determine the frequency of the gastric slow wave so that processor 30 may control the bursts 45 of spike stimulation pulses to be delivered at a predetermined point in the slow wave. Alternatively, as discussed above, processor 30 may be dynamically responsive to a threshold crossing of the sensed gastric slow wave. In some embodiments, each burst 45 of pulses may overlap with a peak of the slow wave 48, while in other embodiments, the bursts may be applied without overlapping with the peaks of the slow wave. The superposition of the pulses over the peak region of the slow wave may more closely simulate the natural wave function of the gastrointestinal tract.

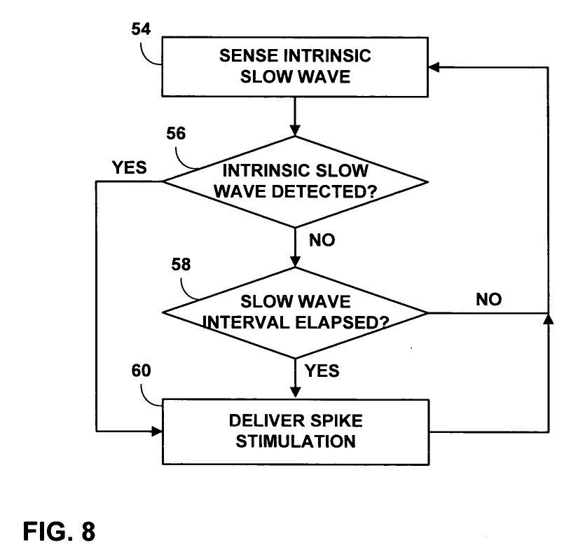

FIG. 8 is a flow chart illustrating a technique for delivery of stimulation pulses in a synchronous burst mode to mimic gastric spike activity. In the example of FIG. 8, implantable stimulator 40 (FIG. 6) senses for the presence of an intrinsic slow wave (54). If an intrinsic slow wave is not detected (56) within a given interval of time (58), stimulator 40 delivers stimulation to mimic the gastric spike activity (60). The sensing (54) and delivery of slow wave stimulation may be performed on a continuous, periodic basis. Likewise, in the event an intrinsic slow wave is detected (56), stimulator 40 delivers spike stimulation (60). The spike stimulation is synchronized to the intrinsic slow wave. As discussed with reference to FIG. 7, for example, the spike stimulation may be delivered in synchronization with a determined frequency of the intrinsic slow wave, or with a threshold crossing of the intrinsic slow wave.

Various embodiments of the described invention may include processors that are realized by microprocessors, Application-Specific Integrated Circuits (ASIC), Field-Programmable Gate Arrays (FPGA), or other equivalent integrated or discrete logic circuitry. The processor may also utilize several different types of data storage media to store computer-readable instructions for device operation. These memory and storage media types may include any form of computer-readable media such as magnetic or optical tape or disks, solid state volatile or non-volatile memory, including random access memory (RAM), read only memory (ROM), electronically programmable memory (EPROM or EEPROM), or flash memory.

In certain embodiments of the invention, gastric slow wave sensing may be accomplished without the need for a separate sensing lead. The stimulation leads may be able to obtain the slow wave information by monitoring electrical signals between stimulation deliveries. A system such as this may be similar to those used in cardiac pacing techniques. The detection of the slow wave may be done using one bipolar stimulation lead or through the use of a combination of multiple of leads. The use of the same stimulation leads may be beneficial to the patient due of fewer leads tunneled through tissue and possible decreases in therapy costs.

In other embodiments, sensing electrical signals may not be limited to the gastric slow wave. The stimulator may be able to monitor gastric spike action potentials during or separate from stimulation therapy. The system may be programmed to monitor the gastric spike action potentials, and stimulation therapy may begin upon sensing inadequate spike action potentials. Alternatively, the system may be able to detect intrinsic spike action potentials during burst therapy in order to suspend stimulation upon sufficient intrinsic stimulation. This type of monitoring may enable a more flexible stimulation therapy capable of improving gastric motility in patients with sporadic interventional needs.

Also, in some embodiments, a sensor may be used exclusively for monitoring the gastric slow wave or gastric spike action potentials without providing feedback for stimulation therapy. In this case, sensed gastric slow wave information may be used to adjust stimulation periodically, rather than dynamically as the slow wave is sensed. In either case, the slow wave may be measured continuously, intermittently or at the direction of an external programmer. The sensed gastric slow wave information may be used for disease diagnosis or condition monitoring and may permit a patient to avoid frequent clinic visits.

Many embodiments of the invention have been described. Various modifications may be made without departing from the scope of the claims. These and other embodiments are within the scope of the following claims.

1. A method for electrical stimulation of a gastrointestinal tract of a patient, the method comprising:
generating electrical stimulation pulses at a rate of approximately 30 to 120 pulses per minute; and
delivering the stimulation pulses to the gastrointestinal tract to regulate gastric motility.

2. The method of claim 1, further comprising applying the pulses substantially continuously.

3. The method of claim 1, further comprising generating the stimulation pulses at a rate of approximately 60 to 90 pulses per minute.

4. The method of claim 1, wherein the stimulation pulses mimic gastric spike activity of the gastrointestinal tract.

5. The method of claim 1, wherein each of the stimulation pulses has an amplitude of approximately 1 to 15 volts.

6. The method of claim 1, further comprising delivering the stimulation pulses in a series of bursts.

7. The method of claim 6, further comprising delivering the bursts at a rate of approximately 2 to 20 bursts per minute.

8. The method of claim 6, further comprising:
sensing gastric slow wave activity; and
delivering the bursts in synchronization with the sensed gastric slow wave activity.

9. A system for electrical stimulation of a gastrointestinal tract of a patient, the system comprising:
a pulse generator that generates electrical stimulation pulses at a rate of 30 to 120 pulses per minute; and
one or more leads that apply the pulses to the gastrointestinal tract to regulate gastric motility.

10. The system of claim 9, wherein the pulse generator generates the pulses substantially continuously.

11. The system of claim 9, wherein the pulse generator generates the stimulation pulses at a rate of approximately 60 to 90 pulses per minute.

12. The system of claim 9, wherein the stimulation pulses mimic gastric spike activity of the gastrointestinal tract.

13. The system of claim 9, wherein the stimulation pulses have an amplitude of approximately 1 to 15 volts.

14. The system of claim 9, wherein the pulse generator generates the stimulation pulses in a series of bursts.

15. The system of claim 14, further comprising delivering the bursts at a rate of approximately 2 to 20 bursts per minute.

16. The system of claim 14, further comprising a sensor that senses gastric slow wave activity, wherein the pulse generator generates the bursts in synchronization with the sensed gastric slow wave activity.

17. A system for electrical stimulation of a gastrointestinal tract of a patient, the system comprising:
means for generating electrical stimulation pulses at a rate of approximately 30 to 120 pulses per minute; and
means for delivering the stimulation pulses to the gastrointestinal tract to regulate gastric motility.

18. The system of claim 17, further comprising means for applying the pulses substantially continuously.

19. The system of claim 17, further comprising means for generating the stimulation pulses at a rate of approximately 60 to 90 pulses per minute.

20. The system of claim 17, wherein the stimulation pulses mimic gastric spike activity of the gastrointestinal tract.

21. The system of claim 1, further comprising means for delivering the stimulation pulses in a series of bursts.

22. The system of claim 21, further comprising means for delivering the bursts at a rate of approximately 2 to 20 bursts per minute.

23. The system of claim 21, further comprising:
means for sensing gastric slow wave activity; and
means for delivering the bursts in synchronization with the sensed gastric slow wave activity.

24. A method for electrical stimulation of a gastrointestinal tract of a patient, the method comprising:
generating electrical stimulation pulses at a rate of approximately 30 to 120 pulses per minute; and
delivering the pulses to the gastrointestinal tract in bursts at a rate of approximately 2 to 20 bursts per minute to regulate gastric motility.

25. The method of claim 24, further comprising generating the stimulation pulses at a rate of approximately 60 to 90 pulses per minute.

26. The method of claim 24, wherein the stimulation pulses mimic gastric spike activity of the gastrointestinal tract.

27. The method of claim 24, further comprising:
sensing gastric slow wave activity; and
delivering the bursts in synchronization with the sensed gastric slow wave activity.
